(19) United States
(12) Patent Application Publication
(10) Pub. No.: US 2017/0360392 A1
(43) Pub. Date: Dec. 21, 2017

(54) Radiation Image Processing System And Radiation Image Processing Apparatus

(71) Applicant: KONICA MINOLTA, INC., Chiyoda-ku (JP)

(72) Inventor: Shikou KANEKO, Niiza-shi (JP)

(73) Assignee: KONICA MINOLTA, INC., Tokyo (JP)

(21) Appl. No.: 15/622,965

(22) Filed: Jun. 14, 2017

(30) Foreign Application Priority Data
Jun. 20, 2016 (JP) 2016-121341

(51) Int. Cl. A61B 6/00

(52) U.S. Cl. CPC A61B 6/5288; A61B 6/503; A61B 6/5211

(57) ABSTRACT

A radiation image processing system, including: an image analysis section which performs image analysis to a plurality of frame images that is obtained by moving image imaging of a target site having periodicity in a movement or frame images that are obtained after image processing based on the frame images obtained by the moving image imaging and which determines a period of the movement; and a reproduction range setting section which sets a range of frame images to be reproduced and displayed among the frame images based on an analysis result of the period determined by the image analysis section.

### CROSS-REFERENCE TO RELATED APPLICATIONS

The entire disclosure of Japanese Patent Application No. 2016-121341 filed on Jun. 20, 2016 including description, claims, drawings and abstract are incorporated herein by reference in its entirety.

### BACKGROUND OF THE INVENTION

### 1. Field of the Invention

The present invention relates to a radiation image processing system and a radiation image processing apparatus.

### 2. Description of Related Art

Attempts have been made to use semiconductor image sensors such as FPDs (flat panel detectors) for performing dynamic imaging (imaging of dynamic states) at sites which are diagnosis targets (hereinafter, referred to as target sites) and use the obtained results for diagnosis instead of capturing and diagnosing still images of radiation (X-ray) using conventional films/screens and stimulable phosphor plates.

Specifically, by using rapid responsiveness of a semiconductor image sensor in reading and deleting image data, pulsed radiation is continuously emitted from a radiation source at the reading and deleting timings of the semiconductor image sensor, and imaging is performed a plurality of times per second to capture a dynamic state at a target site. By sequentially displaying a series of frame images obtained by the dynamic imaging, a doctor can observe a series of movements at the target site.

For example, in a case where the target site is a lung or a heart, the doctor can observe and diagnose the point where a lung function (ventilation function, pulmonary blood flow function or the like) is lowered, the point where heartbeat of the heart is abnormal or the like by watching the manner of movement of the lung or the heart which is reproduced on a screen, that is, watching each of the frame images (see FIG. 11, for example) obtained by dynamic imaging of the lung or the heart (see Japanese Patent Application Laid Open Publication No. 2012-5729, for example).

When moving image reproduction is performed for a plurality of frame images which is obtained by dynamic imaging, in some cases, loop reproduction is performed for the frame images (that is, the moving image is repeatedly reproduced by restarting the moving image reproduction from the first frame image immediately and continuously after the end of display of the last frame image).

At that time, in a case where the target site is a lung of a patient, for example, the lung state of the patient does not change from a resting inspiratory phase to a resting expiratory phase in a moment if the last frame image and the first frame image show entirely different lung states (for example, a resting inspiratory phase and a resting expiratory phase). Thus, the doctor watching the change can recognize that the moving image reproduction of a series of frame images ended and the next moving image reproduction is newly started at the moment when the lung state is switched from the resting inspiratory phase to the resting expiratory phase.

As shown in FIG. 11, in a case where both of the last frame image and the first frame image show the resting inspiratory phases of the lung, for example, when loop reproduction is performed for the plurality of frame images, the moving image reproduction is ended at the resting inspiratory phase and the next moving image reproduction is immediately started at the resting inspiratory phase. Thus, each of the frame images is reproduced as if the patient naturally continues the breathing.

The resting inspiratory phase and the resting expiratory phase respectively indicate the state in which the patient breathes in to make the lung maximum and the state in which the patient breathes out to make the lung minimum when the patient performs breathing in a resting state. The maximum inspiratory phase and the maximum expiratory phase respectively indicate the state in which the patient fully breathes in to make the lung maximum and the state in which the patient fully breathes out to make the lung minimum.

However, in a case where the lung states captured in the last frame image and the first frame image are similar to each other, for example, when loop reproduction is performed for the plurality of frame images, the lung which has been naturally moving moves discontinuously and, for example, appears to twitch unnaturally in some cases at the time when the moving image reproduction is finished and the next moving image reproduction is immediately started.

In such a case, the doctor watching this possibly experiences a feeling of strangeness or possibly misunderstands that the patient has a disease or an abnormality, which decreases the accuracy of diagnosis or leads to wrong diagnosis. When a plurality of frame images obtained by dynamic imaging of a target site having periodicity in a movement of a lung, a heart or the like is reproduced, it is necessary to reproduce the frame images so that the user such as a doctor does not experience a feeling of strangeness, the accuracy of diagnosis by the doctor is not decreased and the wrong diagnosis is not generated.

### SUMMARY OF THE INVENTION

The present invention has been made in consideration of the above problems and an object of the present invention is to provide a radiation image processing system and a radiation image processing apparatus capable of preventing a user from experiencing the feeling of strangeness, decrease in the accuracy of diagnosis and generation of wrong diagnosis at the time of reproducing a plurality of frame images obtained by moving image imaging of a target site which has periodicity in a movement.

In order to solve at least one of the above problems, according to one aspect of a preferred embodiment of the present invention, there is provided a radiation image processing system, including: an image analysis section which performs image analysis to a plurality of frame images that is obtained by moving image imaging of a target site having periodicity in a movement or frame images that are obtained after image processing based on the frame images obtained by the moving image imaging and which determines a period of the movement; and a reproduction range setting section which sets a range of frame images to be reproduced and displayed among the frame images based on an analysis result of the period determined by the image analysis section.

### BRIEF DESCRIPTION OF THE DRAWINGS

The above and other objects, advantages and features of the present invention will become more fully understood from the detailed description given hereinafter and the appended drawings which are given by way of illustration only, and thus are not intended as a definition of the limits of the present invention, and wherein:

FIG. 1 is a block diagram showing a configuration of a radiation image processing apparatus or a radiation image processing system in an embodiment;

FIG. 2 is a view showing a frame image obtained by extracting a signal component of a pulmonary blood flow as an example of each of the frame images obtained after image processing;

FIG. 3A is a view showing positions of a lung apex and such like in each of the frame images;

FIG. 3B is a view showing positions of a heart wall and such like in each of the frame images;

FIG. 4A is a view showing a width, a height and such like of a lung in each of the frame images;

FIG. 4B is a view showing a width, a height and such like of a heart in each of the frame images;

FIG. 5A is a view showing target regions set at positions of a lung apex and such like in each of the frame images;

FIG. 5B is a view showing target regions set at positions of a heart and such like in each of the frame images;

FIG. 6A is a graph plotting a height of a lung as an example of a morphology value;

FIG. 6B is a graph for explaining that a frame image having a same height of a lung L as the height of the lung L in a head frame is set as an end frame;

FIG. 7 is a graph showing an example of a change speed of the morphology value and for explaining how to set the end frame;

FIG. 8 is a view for explaining that a frame image having a morphology value and a change speed of the morphology value which are both same as those of the head frame is set as an end frame;

FIG. 9 is a view showing reproduction and display of each of the frame images in a set range on a display section and display showing how many periods correspond to the range which is under loop reproduction;

FIG. 10 is a view showing display of a seek bar near the frame image which is under loop reproduction or the like; and

FIG. 11 is a view showing an example of each of the frame images obtained by dynamic imaging of a chest of a subject.

### DETAILED DESCRIPTION OF THE PREFERRED EMBODIMENT

Hereinafter, an embodiment of a radiation image processing system and a radiation image processing apparatus according to the present invention will be described with reference to the drawings.

Hereinafter, description will be made for a case where after-mentioned image analysis section and reproduction range setting section are configured in a single apparatus (that is, a case where the radiation image processing apparatus includes the image analysis section and the reproduction range setting section). However, for example, though not shown in the drawings, it is also possible to configure at least the image analysis section and the reproduction range setting section as separate apparatuses and connect the apparatuses via a network or the like to configure a system (that is, as the radiation image processing system).

Hereinafter, description will be made for a case where a display section is formed integrally with the image analysis section and the reproduction range setting section. However, though not shown in the drawings, it is also possible to configure the display section separate from the image analysis section and the reproduction range setting section and connect the sections via a network or the like to configure a system (that is, as the radiation image processing system).

### [Configuration of Radiation Image Processing Apparatus (Radiation Image Processing System)]

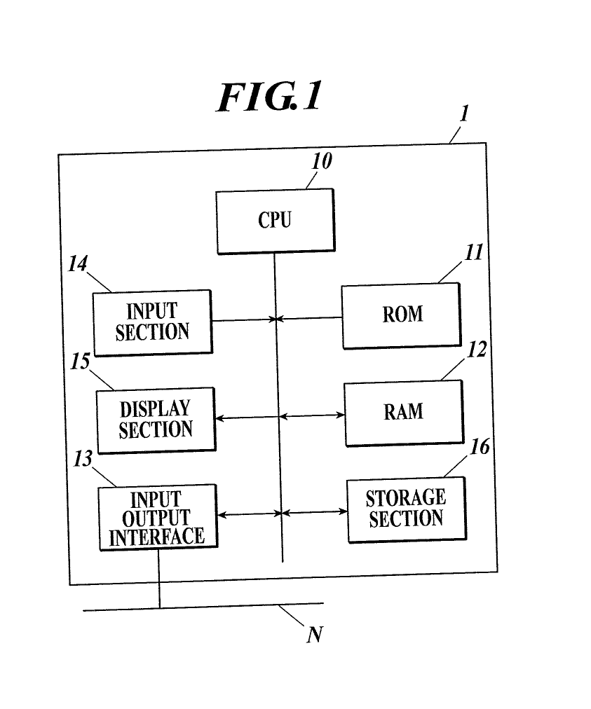

The configuration of a radiation image processing apparatus 1 (or radiation image processing system 1, hereinafter, the same applies) in the embodiment will be described. FIG. 1 is a block diagram showing the configuration of a radiation image processing apparatus in the embodiment. In the embodiment, as shown in FIG. 1, the radiation image processing apparatus 1 is configured by including a general-purpose computer in which a CPU (Central Processing Unit) 10, a ROM (Read Only Memory) 11, a RAM (Random Access Memory) 12, an input output interface 13 and such like are connected to a bus. The radiation image processing apparatus 1 is connected to a network N via the input output interface 13.

Furthermore, the CPU 10 is connected to an input section 14 which is configured by including a keyboard, a mouse, a touch panel and such like, a display section 15 which is configured by including a CRT (Cathode Ray Tube), an LCD (Liquid Crystal Display) or the like, and such like. The CPU 10 is also connected to a storage section 16 which is configured by including a non-volatile semiconductor memory, an HDD (Hard Disk Drive) or the like.

The radiation image processing apparatus 1 can also be configured as a dedicated apparatus, not as a general-purpose computer as described above. Though not shown in the drawings, it is also possible to have a configuration including an image capturing apparatus which performs dynamic imaging, a control apparatus such as a console which controls the image capturing, an image storage database for storing a plurality of frame images which were captured and such like via a network N in addition to the radiation image processing apparatus 1.

In the embodiment, the CPU 10 of the radiation image processing apparatus 1 is configured to function as the image analysis section and the reproduction range setting section according to the present invention, and the display section 15 is configured to function as the display section according to the present invention. Hereinafter, the image analysis section 10, the reproduction range setting section 10 and the display section 15 will be described.

### [Configuration of Image Analysis Section]

Hereinafter, description will be made first for the configuration of the image analysis section 10, that is, processing which is performed in the image analysis section 10. In the embodiment, the image analysis section 10 performs image analysis to each of a plurality of frame images P obtained by moving image imaging of a target site having periodicity in a movement and determines the period τ of the movement. As the method for determining the movement period τ by the image analysis section 10, there can be adopted methods described in International Publication No. 2009/090894, Japanese Patent Application Laid Open Publication No. 2009-273671 and Japanese Patent Application Laid Open Publication No. 2009-153678, for example.

At that time, a plurality of frame images P which is a target of the image analysis may be obtained by moving image imaging of a target site having periodicity in a movement as described above. That is, for example, the frame images P may be obtained by dynamic imaging of a lung, a heart or the like as shown in FIG. 11 (in such a way, dynamic imaging is included in moving image imaging), and though not shown in the drawings, the frame images P may be frame images obtained by moving image imaging capturing a manner in which the patient periodically moves a hand, a leg, a neck, a body or the like (that is, wrist joint, ankle joint, shoulder joint, hip joint or the like) by bending, stretching and twisting, for example.

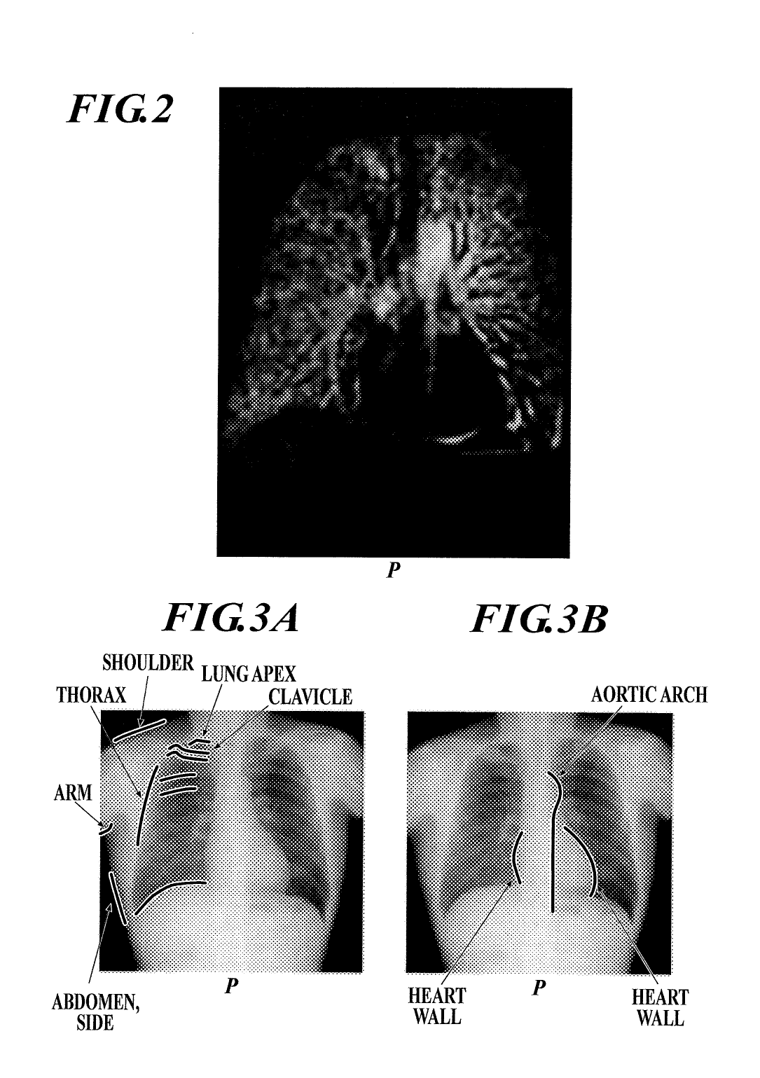

Furthermore, the plurality of frame images P which is a target of the image analysis may be frame images P obtained after image processing such as frame images P (see FIG. 2) obtained by extracting signal components of pulmonary blood flow (blood flow signal components) on the basis of the frame images obtained by dynamic imaging of a lung of a patient as shown in FIG. 2, for example.

In the embodiment, the image analysis section 10 calculates a morphology value α of a body part such as a lung, a heart or the like captured in each of the frame images P and a change speed Δα of the morphology value α.

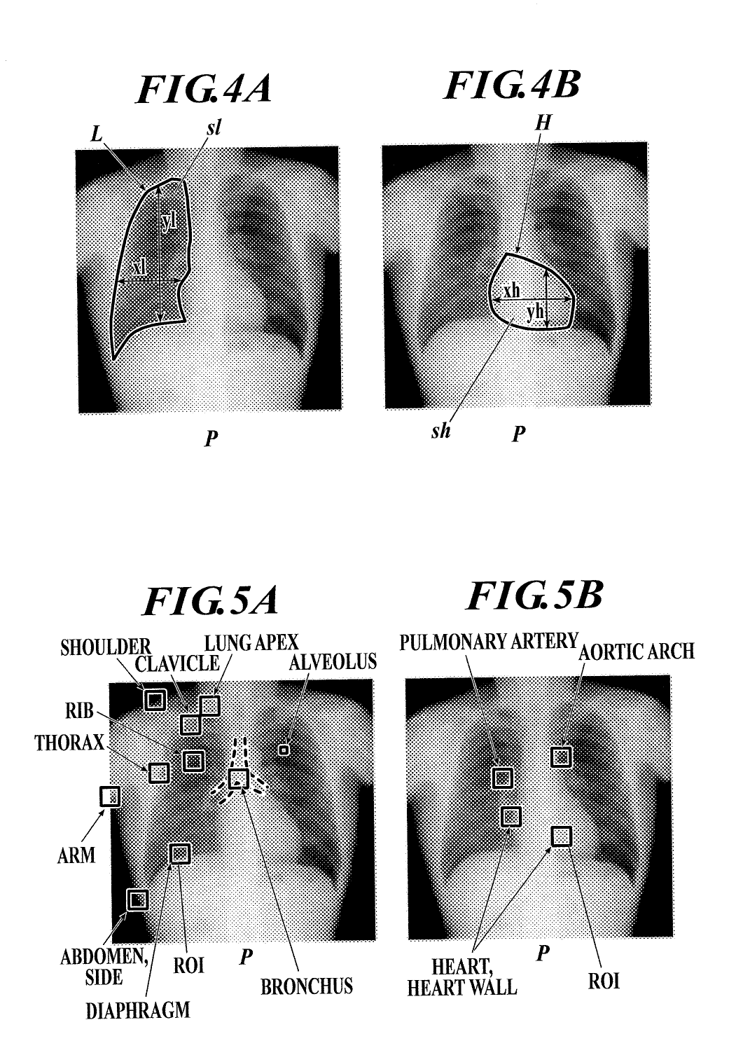

The image analysis section 10 can be configured to calculate any of positions in each of the frame images P of a lung apex, a diaphragm, a rib, a clavicle, a thorax, a shoulder, an arm, an abdomen, a heart wall and an aortic arch as shown in FIGS. 3A and 3B, a width xl, a height yl and an area sl in each of the frame images P of a lung L as shown in FIG. 4A and a width xh, a height yh and an area sh in each of the frame images P of a heart H as shown in FIG. 4B. As the change speed Δα of the morphology value α, for example, any of change speeds Δα of the above-mentioned morphology values α can be calculated.

In the embodiment, the image analysis section 10 sets a target region ROI in each of the frame images P as shown in FIGS. 5A and 5B and calculates a density value β or the change speed Δβ of the density value β of each pixel in the target region ROI. Though FIGS. 5A and 5B show cases of setting rectangular target regions ROI on a frame image P, the target region ROI may be a polygon, a circle, an ellipse or the like, for example, and the shape of the target region ROI is not limited.

As the density value β, the image analysis section 10 can calculate an analysis value (any of average value, median value, mode value, integration value, minimum value and maximum value) of a density value β of each pixel in the target region ROI including a part of any of the lung apex, diaphragm, rib, clavicle, thorax, shoulder, arm, abdomen, heart, alveolus, bronchus, pulmonary artery and aortic arch captured in each of the frame images P.

As the change speed Δβ of the density value β, the change speed of the analysis value of the above density value β can be calculated. Hereinafter, in the embodiment, the density value β and the change speed Δβ of the density value β respectively represent the analysis value of the above density value β and the change speed of the analysis value of the density value β.

### [Configuration of Reproduction Range Setting Section]

Next, the configuration of the reproduction range setting section 10, that is, processing performed in the reproduction range setting section 10 will be described. Hereinafter, the function of the radiation image processing apparatus 1 or the radiation image processing system 1 in the embodiment will be described together.

The reproduction range setting section 10 sets the range of frame images P to be reproduced and displayed on the display section 15 among the frame images P on the basis of the analysis result of the period τ determined by the image analysis section 10.

In the embodiment, the reproduction range setting section 10 sets the range of frame images P to be reproduced and displayed among the plurality of frame images P, which were obtained by moving image imaging, by setting a head frame image Pin in the range (that is, the frame image Pin to be displayed first in the reproduction display, hereinafter, simply referred to as a head frame Pin) and an end frame image Pfi in the range (that is, frame image Pfi to be displayed last in the reproduction display, hereinafter, simply referred to as an end frame Pfi).

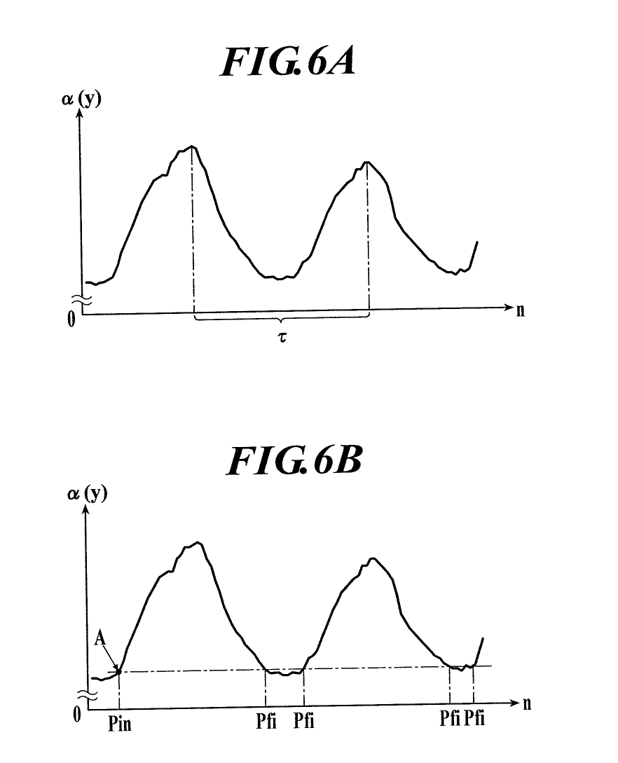

Hereinafter, setting processing of the range of frame images P to be reproduced and displayed by the reproduction range setting section 10 will be specifically described. FIG. 6A is a graph obtained by plotting with the longitudinal axis of the height y (see FIG. 4A) in each of the frame images P of the lung L as an example of the morphology value α and a horizontal axis of a frame number n (n≧1) of each of the frame images P. The τ in FIG. 6A represents a period of breathing.

In a case where the target site is a lung L in such a way, since the doctor cannot perform diagnosis and such like by watching the dynamic state of the lung L unless the frame images P are reproduced for the number of frames of one period or more of the breathing. Thus, the reproduction range setting section 10 sets the range of reproduction and display by targeting the frame images P including at least one period of breathing.

Though not shown in the drawings, in a case where the target site is a heart H (see FIG. 4B and others), for example, the graph of morphology value α regarding the heart H is a graph having periodicity similarly to FIG. 6A. However, the doctor cannot perform diagnosis and such like by watching the dynamic state of the heart H unless the frame images P are reproduced for the number of frames of one period or more of the heartbeat similarly to the case of lung L. Thus, the reproduction range setting section 10 sets the range of reproduction and display by targeting frame images P including at least one period of heartbeat. The same also applies to a case of setting the range of reproduction and display targeting frame images P obtained after image processing as illustrated in FIG. 2.

In the embodiment, the reproduction range setting section 10 sets the above range so that the breathing phase (heartbeat phase in a case where the target site is the heart H, the same applies, hereinafter) of the head frame Pin in the range nearly matches the breathing phase of the end frame Pfi in the range in the graph shown in FIG. 6A, for example.

### [Range Setting Method 1]

At that time, as shown in FIG. 6B, for example, when the point A in the graph is selected as the head frame Pin of the reproduction display range, since the morphology value α is the height y of the lung L in this case, the range can be set by setting a frame image P having a height y of lung L which is same or nearly same as the height y of lung L in the head frame Pin as the end frame Pfi in the range. There are cases where a plurality of end frames Pfi can be set as in a case of FIG. 6B.

### [Range Setting Method 2]

The range can be set by setting the head frame Pin of the reproduction display range and the end frame Pfi in the range also on the basis of the above-mentioned change speed Δα of the morphology value α (that is, change speed Δy of the height y of the lung L which is the morphology value α in the embodiment).

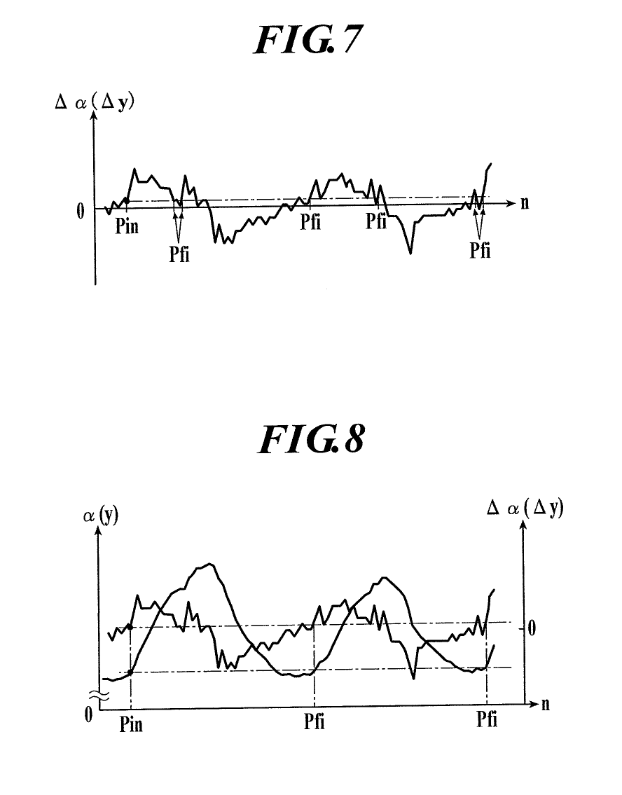

For example, in a case where the morphology value α changes for each of the frame images P as shown in FIG. 6A, FIG. 7 is obtained by plotting the change speeds Δα of the morphology values α (Δy in the above example, that is, the difference between α(y) in a target frame P and α(y) in the previous frame P) for frame numbers n of respective frame images P.

As shown in FIG. 7, when the point A in the graph is selected as the head frame Pin in the reproduction display range, the range can be set by setting, as the end frame Pfi in the range, the frame image P having the same or nearly same change speed Δα of the morphology value α as the change speed Δα (that is, Δy) of the morphology value α in the head frame Pin. Also in this case, there are cases where a plurality of end frames Pfi can be set as shown in FIG. 7.

### [Range Setting Method 3]

As described above, the range of frame images P to be reproduced and displayed can be set by setting the head frame Pin and the end frame Pfi only on the basis of the morphology value α or only on the basis of the change speed Δα of the morphology value α. However, as shown in FIG. 8, for example, the above range can be set by setting, as the end frame Pfi of the reproduction display range, the frame image P having the same or nearly same morphology value α as the morphology value α (that is, y in the above example) in the head frame Pin and having the same or nearly same change speed Δα of the morphology value α as change speed Δα (that is, Δy in the above example) of the morphology value α in the head frame Pin.

In this case, as the end frame Pfi, there is set a frame image P having the same or nearly same height y (that is, morphology value α) of the lung L as the height y of lung L in the head frame Pin and having the same or nearly same change speed (that is, change speed of the morphology value α) of the height y of the lung L. That is, as shown in FIG. 8, for example, as the end frame Pfi, there is set a frame image P capturing the lung L starting to increase the height y similarly to the head frame Pin capturing the lung L starting to increase the height y.

Thus, by such a configuration, it is possible to set the end frame Pfi with more accuracy and accurately set, as the end frame Pfi in the range when reproducing and displaying a plurality of frame images P obtained by moving image imaging of a target site having periodicity in a movement, the frame image P in which the target site moves (expansion, deflation or the like) similarly to the head frame Pin (the morphology of target site is changing similarly).

For example, when loop reproduction is performed for frame images P in the range, the movements of lung L are displayed so as to be naturally connected even if the head frame Pin is reproduced following the end frame Pfi. Thus, it is possible to reproduce (loop reproduction) the frame images P accurately without making the user such as a doctor experience a feeling of strangeness, decreasing the accuracy in diagnosis or generating wrong diagnosis when reproduction or loop reproduction is performed for the frame images P.

Though the above description is made for a case where the target site is a lung L, the same configuration is also possible for cases where the target site is a heart H, a hand, a leg, a neck and such like. Though the above description is made for a case where the reproduction range setting section 10 sets the range of frame images P to be reproduced and displayed on the basis of the morphology value α and/or the change speed Δα of the morphology value α, the same configuration as the above configuration is also possible for a case where the reproduction range setting section 10 sets the range of frame images P to be reproduced and displayed on the basis of the above-mentioned density value β and/or the change speed of the density value β.

Furthermore, even when the frame images P are the frame images P obtained after image processing as shown in FIG. 2, the same configuration is possible as long as the frame images P obtained after image processing are regarding a target site having periodicity in a movement.

[Processing in a Case where there is a Plurality of Candidates for Frame Image P to be Set]

As described above, in a case where the head frame Pin is selected, the reproduction range setting section 10 automatically sets the end frame Pfi and sets the range of frame images P to be reproduced and displayed. However, though the description is omitted, in a case where the end frame Pfi is selected, the reproduction range setting section 10 similarly automatically sets the head frame Pin and sets the range of frame images P to be reproduced and displayed.

At that time, as shown in FIG. 8, for example, in a case where there are a plurality of candidates for the end frame Pfi, when the end frame Pfi (end frame fi in the left side in FIG. 8) which was captured earlier is set as the end frame Pfi, the frame images P for the amount of one period from the head frame Pin to the end frame Pfi are reproduced and displayed when frame images P are reproduced and displayed.

In this case, when the end frame Pfi (end frame fi in the right side in FIG. 8) which was captured later is set, for example, the frame images P for the amount of two periods from the head frame Pin to the end frame Pfi are reproduced and displayed when frame images P are reproduced and displayed.

The reproduction display (loop reproduction) of frame image P is performed for a doctor to see the frame images P and determine whether the patient has abnormality or a disease at the target site as described above, and the abnormality and the disease are possibly captured clearly in a later period. Thus, the range of frame images P to be reproduced and displayed may be a range including as many periods τ as possible.

Thus, in a case where there is a plurality of candidates for the end frame Pfi in the range of frame images P to be reproduced and displayed, it is preferable that the reproduction range setting section 10 sets the end frame Pfi which was captured later as the end frame Pfi in the range.

When the end frame Pfi is selected, in a case where there is a plurality of candidates for the head frame Pin in the range of frame images P to be reproduced and displayed, it is preferable that the reproduction range setting section 10 sets the head frame Pin which was captured earlier as the head frame Pin in the range for the same reason as the above case.

### [How to Select Head Frame Pin and End Frame Pfi]

In the embodiment, as described above, when the head frame Pin or the end frame Pfi is selected, the reproduction range setting section 10 sets the end frame Pfi or the head frame Pin as described above and sets the range of frame images P to be reproduced and displayed.

The head frame Pin and the end frame Pfi may be selected by a doctor, a radiologist or the like who is a user selecting arbitrary one of the plurality of frame images P, and may be selected on a graph such as FIGS. 6B, 7 and 8, for example. For example, in a case where the target site is the lung L, the reproduction range setting section 10 may display “resting inspiratory phase (maximum inspiratory phase)”, “resting expiratory phase (maximum expiratory phase)”, “intermediate phase” and such like on the display section 15 so that the user selects one of them.

The reproduction range setting section 10 may automatically select a frame image P at the resting inspiratory phase or the resting expiratory phase or an arbitrary frame image P. At that time, for example, the reproduction range setting section 10 may refer to the disease of the patient to automatically select the head frame Pin and the end frame Pfi.

That is, though the same also applies to a case of selecting by a user, for example, in a case where the disease of patient is a restrictive disease such as pneumonia and pulmonary fibrosis, emphysema, atelectasis or the like, since the ability to take air into lungs is decreased, there may be selected, as the head frame Pin or the end frame Pfi, a frame image P at or near the phase from which air is taken into the lungs such as the resting expiratory phase (for example, see FIG. 11) and maximum expiratory phase.

By such a configuration, since the frame images P are reproduced (loop reproduction) from a phase from which the air is taken into lungs of the patient, the doctor easily evaluates the ventilation function such as the difficulty in taking air into lungs by watching the reproduced frame images P.

Also, for example, in a case where the disease of patient is an obstructive disease such as asthma and chronic obstructive pulmonary disease (COPD), since the ability to take air out from lungs is decreased, there may be selected, as the head frame Pin or the end frame Pfi, a frame image P at or near the phase from which air is taken out from the lungs such as the resting inspiratory phase (for example, see FIG. 11) and maximum inspiratory phase.

By such a configuration, since the frame images P are reproduced (loop reproduction) from a phase from which the air is taken out from lungs of the patient, the doctor easily evaluates the ventilation function such as the difficulty in taking air out from lungs by watching the reproduced frame images P.

### [Reproduction and Display on Display Section]

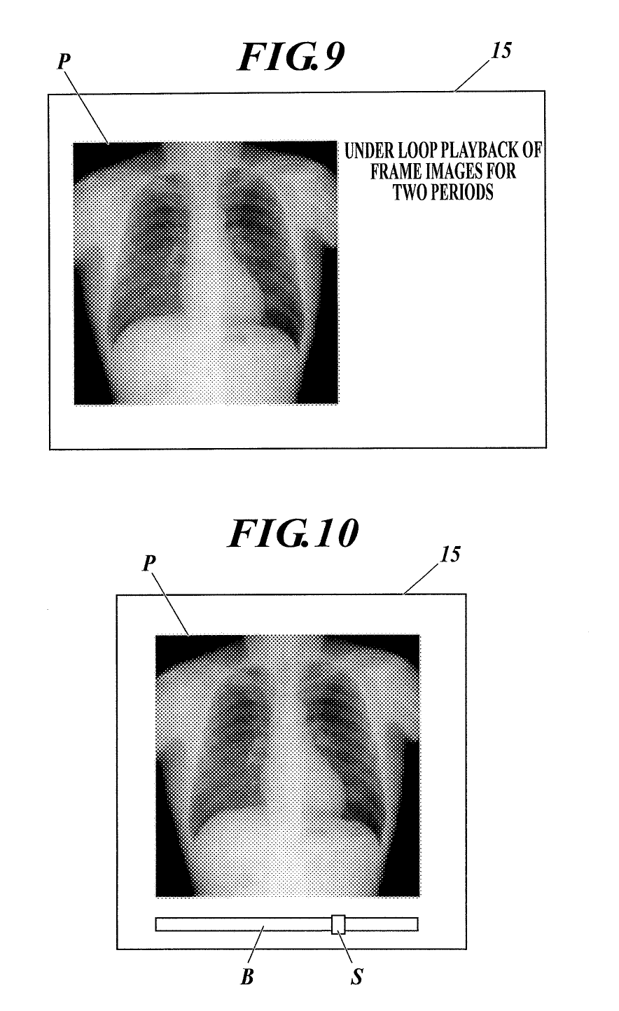

In the embodiment, the frame images P in the range (that is, from the head frame Pin to the end frame Pfi) which was set by the reproduction range setting section 10 as described above are reproduced and displayed on the display section 15 as shown in FIG. 9. As mentioned above, the display section 15 may be separately configured from the image analysis section 10 and the reproduction range setting section 10.

Though each of the frame images P may be displayed once on the display section 15 from the head frame Pin to the end frame Pfi, the frame images P from the head frame Pin to the end frame Pfi may be reproduced by loop reproduction to be displayed.

By such a configuration, loop reproduction is performed to frame images P in a state in which the end frame Pfi of a loop and the head frame Pin of the next loop are smoothly connected to each other. This avoids the state in which the lung L or the like which is being reproduced and displayed appears to discontinuously move and unnaturally twitch. Thus, loop reproduction can be performed in a state in which the lung L naturally moves.

In a case where the lung L or the like unnaturally moves due to a disease or the like, the unnatural movement is truly reproduced in the embodiment. That is, the radiation image processing system 1 or the radiation image processing apparatus 1 in the embodiment prevents the target site from moving unnaturally at the connection part of frames when performing loop reproduction by connecting the end frame Pfi of a loop to the head frame Pin of the next loop. In a case where the target site has an unnatural movement due to a disease or the like, the radiation image processing system 1 or the radiation image processing apparatus 1 in the embodiment does not remove the unnatural movement when performing reproduction and display.

In the embodiment, since the end frame Pfi of a loop is naturally connected to the head frame Pin of the next loop as described above, the doctor or the like watching it possibly loses the number of periods of frame images P under loop reproduction and repeated display.

Thus, as shown in FIG. 9, for example, the display section 15 may display the number of periods corresponding to the range under loop reproduction by displaying, for example, “under loop playback of frame images for two periods” simultaneously with reproduction of the frame images P. By such a configuration, the doctor or the like can accurately recognize the number of periods of frame images P under loop reproduction and repeated display.

### [Effect]

As described above, according to the radiation image processing system 1 and the radiation image processing apparatus 1 in the embodiment, the image analysis section 10 performs image analysis to a plurality of frame images P (or frame images P (see FIG. 2) obtained after image processing based on the plurality of frame images P) obtained by moving image imaging of a target site having periodicity in a movement, and the reproduction range setting section 10 sets the range of frame image P to be reproduced and displayed among the frame images P on the basis of the analysis result of period T determined by the image analysis section 10.

When a plurality of frame images P obtained by moving image imaging of a target site (lung L, heart H, hand, leg, neck or the like) having periodicity in a movement is reproduced, the movements of the target site are displayed so as to be connected to each other naturally and smoothly. Thus, when the frame images P are reproduced or loop reproduction is performed for the frame images P, the user such as a doctor does not experience a feeling of strangeness, the accuracy of diagnosis is not decreased and wrong diagnosis is not generated, and thus, the frame images P can be accurately reproduced (loop reproduction can be accurately performed).

### Modification Examples

In the embodiment, as described above, the image analysis section 10 and the reproduction range setting section 10 read out a plurality of frame images P obtained by moving image imaging from an image storage database or the like, once store the frame images P in the storage section 16 (see FIG. 1) or the like and perform the above processing. After the head frame Pin and the end frame Pfi are set as described above, the frame images P before the head frame Pin and frame images P after the end frame Pfi are not necessary.

Thus, after the head frame Pin and the end frame Pfi are set as described above, the frame images P before the head frame Pin and frame images P after the end frame Pfi may be deleted from the storage section 16 and removed from the target of processing. By such a configuration, unnecessary frame images P are deleted and the data amount can be reduced.

In a case where the frame rate of moving image imaging is less than 10 fps (especially less than 5 fps), when the frame images P are reproduced and displayed, the frame images P are reproduced as in frame advance, and the target site does not appear to smoothly move. Thus, when loop reproduction is performed for such frame images P, for example, the target site such as lung L and heart H appears to twitch or discontinuously move each time the frame is switched. Thus, the doctor or the like does not experience a feeling of strangeness since he/she sees the moving image as such.

Thus, there may be a configuration of not applying the present invention to frame images of such a moving image, that is, a moving image captured at a frame rate of less than 10 fps (or less than 5 fps). There is no sense in applying the present invention to such frame images P since the movements of the target site are not smoothly connected to each other between the end frame Pfi and the head frame Pin of the next loop. By such a configuration, the burden on the reproduction range setting section 10 and such like can be reduced for the amount of reduced wasteful processing.

Furthermore, the image analysis section 10, the reproduction range setting section 10 and such like may perform the above processing only when instructed by a doctor or the like who is a user. By such a configuration, the burden on the reproduction range setting section 10 and such like can be reduced.

Along with the above embodiment, or independently from the above embodiment, the following configuration, for example, may be also applied for making the user such as a doctor recognize that that the loop reproduction returned to a first frame image P of the loop when loop reproduction is performed for frame images P. In a case where the following configuration is applied to the embodiment, the “first frame image P of the loop” and the “last frame image P of the loop” in the following description respectively indicate the above “head frame Pin” and “end frame Pfi”. However, in a case where the following configuration is applied independently from the embodiment, the “first frame image P of the loop” and the “last frame image P of the loop” in the following description may not necessarily be the above “head frame Pin” and the “end frame Pfi”.

For example, when loop reproduction is performed for the frame images P in a range on the display section 15, the portion around the frame image P is displayed so as to be colored, and the color around the frame image P gradually changes as the frame image P to be displayed is switched from the first frame image P of the loop to the last frame image P of the loop. By setting the first frame image P of the loop and the last frame image P of the loop to have entirely different colors (colors not similar to each other), when the loop reproduction is performed, the color around the frame image P changes to an entirely different color at the time when the loop reproduction returns to the first frame image P of the loop. Thus, the user can recognize that the loop reproduction returned to the first frame image P of the loop.

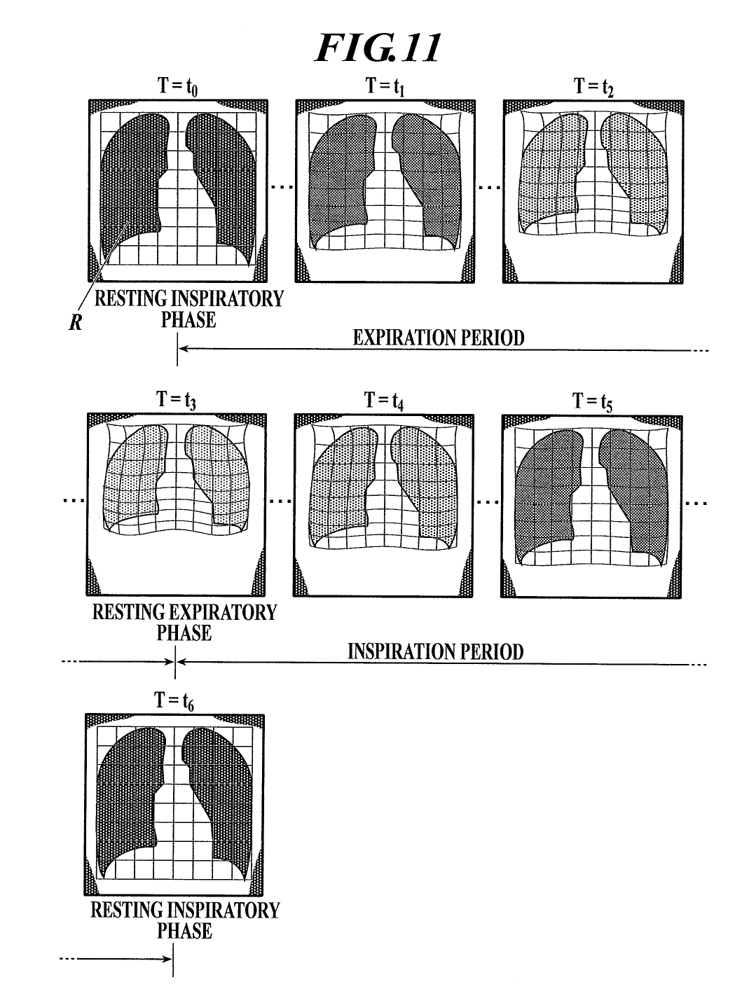

After the last frame image P of a loop is displayed, sound may be made at the time when the first frame image P of the next loop is displayed. Furthermore, a progress bar may be displayed near the frame image P under loop reproduction or a seek bar B may be displayed as shown in FIG. 10. At that time, the color, size and such like of a slider S may be changed in accordance with the progress of loop reproduction.

Furthermore, after the last frame image P of a loop is displayed, at the time when the first frame image P of the next loop is displayed, an interval (temporary stop, decrease in reproduction speed or the like) may be provided in loop reproduction. By such a configuration, the user can surely recognize that the loop reproduction returned to the first frame image P of the loop.

The present invention is not limited to the above embodiment, modification examples and such like, and changes can be appropriately made within the scope of the present invention.

What is claimed is:

1. A radiation image processing system, comprising:
an image analysis section which performs image analysis to a plurality of frame images that is obtained by moving image imaging of a target site having periodicity in a movement or frame images that are obtained after image processing based on the frame images obtained by the moving image imaging and which determines a period of the movement; and
a reproduction range setting section which sets a range of frame images to be reproduced and displayed among the frame images based on an analysis result of the period determined by the image analysis section.

2. The radiation image processing system according to claim 1, wherein the reproduction range setting section sets the range so that the range is a range of frame images which are obtained by dynamic imaging of a chest of a subject or frame images which are obtained after image processing based on the frame images obtained by the dynamic imaging, the range includes at least one period of breathing, and a breathing phase of a head frame image in the range nearly matches a breathing phase of an end frame image in the range.

3. The radiation image processing system according to claim 1, wherein the reproduction range setting section sets the range so that the range is a range of frame images which are obtained by dynamic imaging of a chest of a subject or frame images which are obtained after image processing based on the frame images obtained by the dynamic imaging, the range includes at least one period of heartbeat, and a heartbeat phase of a head frame image in the range nearly matches a heartbeat phase of an end frame image in the range.

4. The radiation image processing system according to claim 2, wherein
when there is a plurality of candidates for the end frame image in the range, the reproduction range setting section sets a frame image which is captured later to be the end frame image in the range, and
when there is a plurality of candidates for the head frame image in the range, the reproduction range setting section sets a frame image which is captured earlier to be the head frame image in the range.

5. The radiation image processing system according to claim 2, wherein the reproduction range setting section sets the head frame image in the range and the end frame image in the range based on a morphology value of a body part captured in each of the frame images, a change speed of the morphology value or both of the morphology value and the change speed of the morphology value.

6. The radiation image processing system according to claim 5, wherein the morphology value is any of positions of a lung apex, a diaphragm, a rib, a clavicle, a thorax, a shoulder, an arm, an abdomen, a heart wall and an aortic arch in each of the frame images, a width, a height and an area of a lung in each of the frame images, and a width, a height and an area of a heart in each of the frame images.

7. The radiation image processing system according to claim 5, wherein the change speed of the morphology value is any of change speeds of positions of a lung apex, a diaphragm, a rib, a clavicle, a thorax, a shoulder, an arm, an abdomen, a heart wall and an aortic arch in each of the frame images, a change speed of a width, a change speed of a height and a change speed of an area of a lung in each of the frame images, and a change speed of a width, a change speed of a height and a change speed of an area of a heart in each of the frame images.

8. The radiation image processing system according to claim 2, wherein the reproduction range setting section sets the head frame image in the range and the end frame image in the range based on a density value of a pixel in each of the frame images, a change speed of the density value or both of the density value and the change speed of the density value.

9. The radiation image processing system according to claim 8, wherein the density value is an analysis value of a density value of each pixel in a target region which includes a part of any of a lung apex, a diaphragm, a rib, a clavicle, a thorax, a shoulder, an arm, an abdomen, a heart, an alveolus, a bronchus, a pulmonary artery and an aortic arch captured in each of the frame images.

10. The radiation image processing system according to claim 8, wherein the change speed of the density value is a change speed of an analysis value of a density value of each pixel in a target region which includes a part of any of a lung apex, a diaphragm, a rib, a clavicle, a thorax, a shoulder, an arm, an abdomen, a heart, an alveolus, a bronchus, a pulmonary artery and an aortic arch captured in each of the frame images.

11. The radiation image processing system according to claim 9, wherein the analysis value is any of an average value, a median value, a mode value, an integration value, a minimum value and a maximum value of the density value of each pixel in the target region.

12. The radiation image processing system according to claim 1, further comprising a display section which displays the frame images in the range set by the reproduction range setting section by reproducing the frame images.

13. The radiation image processing system according to claim 12, wherein the display section displays the frame images in the range by performing loop reproduction of the frame images.

14. The radiation image processing system according to claim 13, wherein the display section displays a number of periods corresponding to the range simultaneously with reproduction of the frame images.

15. A radiation image processing apparatus, comprising:
an image analysis section which performs image analysis to a plurality of frame images that is obtained by moving image imaging of a target site having periodicity in a movement or frame images that are obtained after image processing based on the frame images obtained by the moving image imaging and which determines a period of the movement; and
a reproduction range setting section which sets a range of frame images to be reproduced and displayed among the frame images based on an analysis result of the period determined by the image analysis section.
